(19) United States
(12) Patent Application Publication
(10) Pub. No.: US 2018/0214064 A1
(43) Pub. Date: Aug. 2, 2018

(54) Neuroimaging Headset

(71) Applicant: York Instruments Limited, Heslington, York (GB)

(72) Inventor: Victor Tikhonovich Petrashov, Egham, Surrey (GB)

(73) Assignee: York Instruments Limited, Heslington, York (GB)

(21) Appl. No.: 15/747,212

(22) PCT Filed: Jul. 27, 2016
(86) PCT No.: PCT/GB2016/052295

§ 371 Date: Jan. 24, 2018

(30) Foreign Application Priority Data
Jul. 27, 2015 (GB) 1513191.5

(51) Int. Cl. A61B 5/00; G01R 33/035; G01R 33/00; A61B 5/04

(52) U.S. Cl. CPC A61B 5/4064; G01R 33/0354; G01R 33/0047; A61B 2562/04; A61B 5/6803; A61B 2562/0223; A61B 2562/0271; A61B 5/04008

(57) ABSTRACT

There is described a neuroimaging apparatus in which a plurality of sensing devices, e.g. SQUIDs, are accommodated in a temperature-controlled chamber e.g. a dewar, has a detachable headset including a plurality of pick-up devices. The pick-up devices are arranged in the detachable headset to conform to a body part of a measurement subject. A plurality of different detachable headsets can have pick-up devices arranged for use with different body parts.

### FIELD OF THE INVENTION

The present invention relates to apparatus for neuroimaging and in particular to headsets for such apparatus.

### BACKGROUND OF THE INVENTION

Neuroimaging refers generally to the imaging of parts of the human or animal nervous system, especially the brain, to obtain information about the structure or function thereof. One neuroimaging technique is magnetoencephalography (MEG). In MEG the magnetic fields produced by electrical activity in the brain are measured. This requires extremely sensitive devices such as superconducting quantum interference devices (SQUIDs). MEG can provide a more direct measurement of neural electrical activity compared to functional MRI (fMRI) with very high temporal resolution.

The SQUIDs, which are necessary to measure the extremely low magnetic fields that are generated by the brain, must be kept at a very low temperature, e.g. about 4.2 K, so that they are superconducting. The SQUIDs must be kept in a dewar (vacuum flask) which is cooled using liquid helium and may include parts in a vacuum. In spite of their sensitivity, the signal coils coupled to the SQUIDs need to be very close, e.g. within a few mm, to the scalp in order to detect the magnetic fields of interest. The dewar is necessarily bulky and rigid.

It is very difficult to construct an apparatus that can position the SQUIDs sufficiently close to the scalp whilst maintaining the SQUIDs at a sufficiently low temperature for superconductivity to occur and insulating the patient from that low temperature.

### SUMMARY OF THE INVENTION

It is an aim of the invention to provide an improved headset for a neuroimaging device that can at least partially solve at least one problem of the prior art.

According to the present invention there is provided a neurological imaging apparatus comprising:
a temperature-controlled chamber;
a plurality of sensing devices accommodated in the temperature-controlled chamber;
a head unit detachably mountable to the temperature-controlled chamber;
a plurality of pick-up devices for picking up respective measurement signals mounted in the head unit; and
an interface for communicating the measurement signals to the sensing devices.

According to the present invention there is provided a neurological imaging apparatus comprising:
a temperature-controlled chamber;
a plurality of sensing devices accommodated in the temperature-controlled chamber; and
an interface for receiving a detachable head unit and communicating measurement signals therefrom to the sensing devices.

According to the present invention there is provided a head unit comprising a plurality of pick-up devices and configured for use with an apparatus as described above.

According to the present invention there is provided a neurological imaging method using a neurological imaging apparatus having detachable head units, the method comprising:
selecting a head unit suitable for imaging a body part of a measurement subject;
mounting the head unit to the neurological imaging apparatus; and
imaging the body part of the measurement subject

### BRIEF DESCRIPTION OF THE DRAWINGS

Exemplary embodiments of the present invention are described further below with reference to the accompanying drawings, in which:

FIG. 1 depicts a neuroimaging apparatus including a detachable headset according to an embodiment of the present invention;

FIG. 2 depicts a detachable headset for use in the neuroimaging apparatus of FIG. 1;

FIG. 3 depicts another detachable headset for use in the neuroimaging apparatus of FIG. 1;

FIG. 4 depicts an interface plate for connecting a detachable headset to a neuroimaging apparatus;

FIG. 5 depicts another interface plate for connecting a detachable headset to a neuroimaging apparatus; and

FIG. 6 is a circuit diagram of a pick-up circuit.

In the various drawings, like parts are indicated by like references.

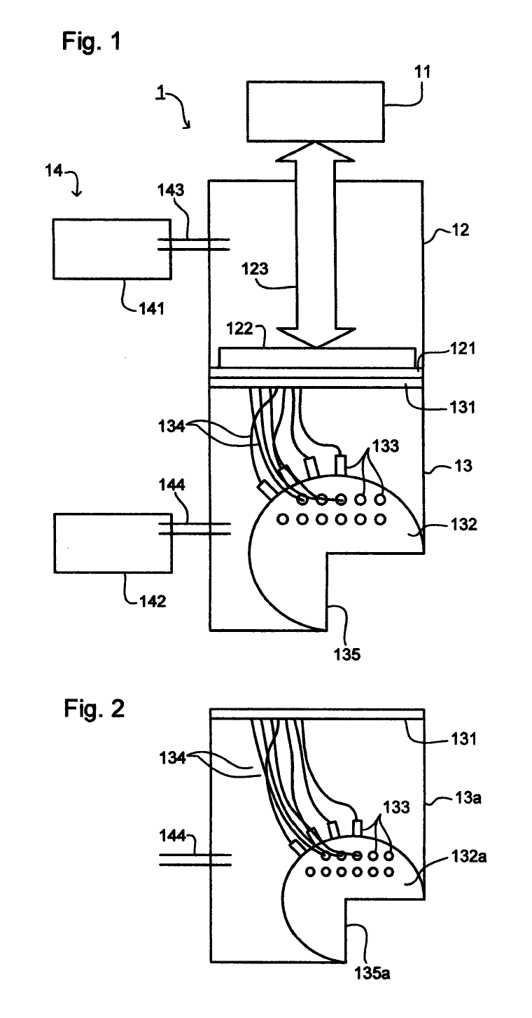

A neuroimaging apparatus 1 according to an exemplary embodiment of the invention is depicted schematically in FIG. 1. Its major subsystems are a control system 11, a dewar 12, a detachable headset 13 and a cooling system 14. Dewar 12 is cooled by coiling system 14 so as to form a temperature-controlled chamber.

Control system 11 controls operation of the apparatus as a whole and may comprise one or more suitably programmed general purpose computers. Control system 11 may simply obtain measurement data which is passed to another system for recording and analysis or may itself record and/or analyse the obtained measurement signals.

Dewar 12 accommodates a SQUID array 122 and a measurement data bus 123 for communicating measurement data to control system 11. Other electronics, such as amplifiers and analogue to digital converters may also be contained within dewar 12. Cooling system 14 includes first cooling unit 141 which communicates via the first cooling duct 143 with dewar 12 so as to cool the interior of dewar 12 to a temperature below the critical temperature T_{c} of the SQUIDs. For example, first cooling unit 141 may supply liquid helium to the interior of dewar 12 at a temperature of less than 4.5 K.

Dewar 12 also includes dewar interface plate 121 for connection to detachable headset 13. Detachable headset 13 has a headset interface plate 131 adapted for connection to the chamber interface plate 121 of the dewar 12. Dewar interface plate 121 and headset interface 131 together form an interface between the dewar 12 and headset 13. The interface provides a mechanical interconnection and a path for measurement signals.

Detachable headset 13 includes a cap 132 which defines a cavity 135 open to the exterior that is shaped to receive the head of a measurement subject. Cap 132 supports a plurality of pick-up coils 133 which are adapted to pick-up magnetic fields generated by neurological activity in the measurement subject. Pick-up coils 133 can also be referred to as signal coils. Cap 132 also includes thermal insulation to protect the measurement subject from the low temperatures at which the pick-up coils are maintained.

The number of pick-up coils 133 depends on the desired spatial resolution of the measurements to be made. The number of pick-up coils 133 may be in the range of from 100 to 500, e.g. in the range of from 200 to 300. Pick up coils 133 are arranged as desired so as to pick up the magnetic fields of interest and desirably surround relevant areas of the measurement subject's head. Conductors 134 are provided to connect pick-up coils 133 to the headset interface plate 131 so that signals can be conveyed to the SQUID array 122.

Second cooling unit 142 is connected to the interior of detachable headset 13 via second conduit 144. To enable a very small magnetic fields to be detected, pick-up coils 133 and conductors 134 are desirably superconducting. However, pick-up coils 133 and conductors 134 can be formed of materials which are superconducting at higher temperatures than those used to form the SQUID so that it may not be necessary to cool detachable headset 13 to a temperature as low as the temperature inside dewar 12. In an embodiment, the interior of detachable headset 13 is coded using liquid nitrogen to a temperature of about 77K.

A further advantage of detachable headset 13 is that a neuroimaging apparatus 1 can be provided with a plurality of detachable headsets 13. The additional headsets may be configured for different measurements. For example, a detachable headset 13a as shown in FIG. 2 may have a smaller cap 132a having a smaller cavity 135a suitable for accommodating a smaller head, e.g. of a child rather than an adult. A measurement apparatus 1 may have a set of detachable headsets 13 each having a cap 132 of a different size so as a whole range of head sizes can be accommodated.

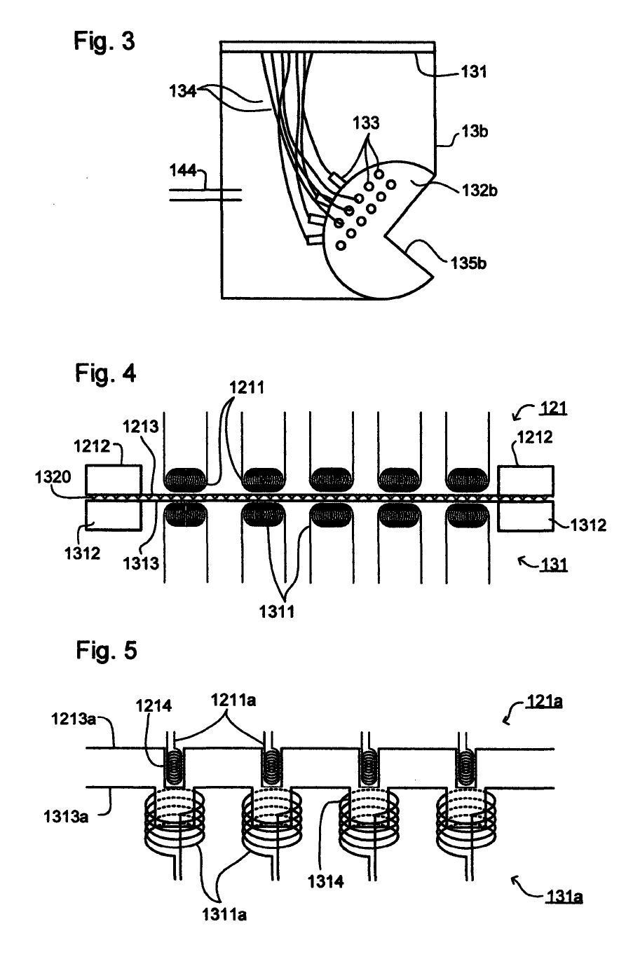

Detachable headsets that differ in other ways can also be provided. For example, FIG. 3 depicts a detachable headset 13b in which the cap 132b is oriented at a different angle so that the cavity 135b can, for example, accommodate the head of a prone measurement subject rather than an upright or seated measurement subject. Detachable headsets 13 may also differ in other respects, e.g. in the number and arrangement of pick-up coils 133.

A detachable headset 13 can be configured for imaging other measurement subjects. For example, a detachable headset 13 can be configured to enable measurements to be made of a foetus in utero. In that case, the cap is configured so as to conform to the abdomen of a pregnant woman, and in particular the fundus of the uterus. Different sizes and/or configurations of detachable headsets can be provided for use in different stages of pregnancy and/or different foetal orientations or numbers.

A detachable headset 13 can be configured to make measurements of non-human animals.

FIG. 4 depicts the interface between dewar 12 and detachable headset 13. Dewar interface plate 121 comprises a plurality of receiver coils 1211 mounted on dewar plate member 1213. Dewar plate member 1213 may form part of the outer wall of dewar 12. Headset interface plate 131 comprises a plurality of transmitter coils 1311 mounted on headset plate member 1313. Headset plate member 1313 may form part of the outer wall of headset 13. The number and arrangement of transmitter coils 1311 and receiver coils 1211 are the same so that when the first plate 1213 and second plate 1313 are joined together via cooperating connectors 1212 and 1312, each transmitter coil 1311 is paired with a respective receiver coil 1211. The transmitter coils 1311 and receiver coils 1211 as well as their relative arrangement when the detachable headset 13 is in place are configured so that the coupling coefficient and mutual inductance between them are as high as possible. Thermal insulation 1320 is provided between dewar interface plate 121 and headset interface plate 131.

Because the interface between dewar 12 and detachable headset 13 is physically remote from the cap 132 and pick-up coils 133, the arrangement of transmitter coils 1311 is not constrained by the arrangement of the pick-up coils 133 on the cap. Therefore, the arrangement of transmitter coils 1311 can be optimised to maximise coupling of the measurement signals and minimise cross talk between measurement channels.

Also, in the event that different superconducting materials are used in the detachable headset 13 than in the dewar 12, so that detachable headset 13 and dewar 12 are maintained at different temperatures, the transmitter coils 1311 and receiver coils 1211 can be configured to transmit the measurement signals across an insulating gap.

FIG. 5 depicts an alternate arrangement of the interface between dewar 12 and detachable headset 13. In this arrangement dewar interface 121a comprises dewar interface plate 1213a which has a plurality of protrusions 1214. The receiver coils 1211a are accommodated within protrusions 1214. The headset interface 131a has headset interface plate 1313a which includes a plurality of recesses 1314 corresponding to the projections 1214. Transmitter coils 1311a are arranged around recesses 1314. With this arrangement, coupling between the transmitter coils 1311a and receiver coils 1211a can be increased. The arrangement can be reversed, i.e. a plurality of projections are provided on the headset interface plate 1313a and a corresponding plurality of recesses are provided on the dewar interface plate 1213a. It is also possible to provide a mixed arrangement, i.e. some measurement channels have the projection on the headset side of the interface and some have the projections on the dewar side of the interface.

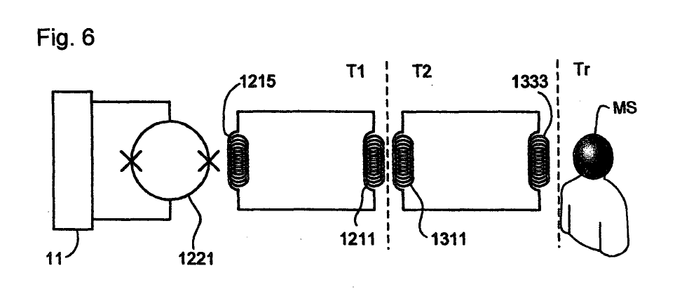

FIG. 6 is a circuit diagram of a measurement channel of a neuroimaging apparatus of an embodiment of the invention. On the right hand side of the figure pick-up coil 133 is positioned in proximity to the measurement subject's head and within the magnetic field to be measured. Changes in the magnetic field experienced by pick-up coil 133 result in the generation of currents therein. Pick-up coil 133 is connected in series with transmitter coil 1311 so that currents generated in pick-up coil 133 flow through transmitter coil 1311 resulting in the generation of a corresponding magnetic field. Desirably pick-up coil 133 and transmitter coil 1311 are superconducting and located in a superconducting circuit so that there are no losses.

Receiver coil 1211 is located on the dewar side of the interface but is electromagnetically coupled with transmitter coil 1311. Therefore the magnetic field generated by transmitter coil 1311 induces a current in receiver coil 1211. Receiver coil 1211 is connected in series with output coil 1215 so that the current induced in receiver coil 1211 passes through output coil 1215. Output coil 1215 generates a magnetic field in response to the current flowing there through. All the coils, including transmitter coil 1211 and output coil 1215 are superconducting and connected by superconducting wires. Output coil 1215 is located adjacent SQUID 1221 or other magnetic sensing device. An electrical signal is generated in SQUID 1221 in response to the magnetic field generated by output coil 1215.

The inductive coupling between transmitter coil 1311 and receiver coil 1211 and between output coil 1215 and SQUID 1221 can be designed to optimise sensitivity of the magnetometer.

An advantage of the invention is that the dewar 12, headset 13 and measurement subject MS can be maintained at different temperatures, T1, T2 and Tr respectively. In an embodiment T1 is about 4 to 5 K, T2 is about 50 to 80 K and Tr is about 300 K. Therefore, the temperature difference Tr−T2, between the environment of the measurement subject and the interior of the headset, is about 220 to 250 K, rather than about 295 K in conventional MEG systems. Hence the signal coils can be placed closer to the measurement signal for a given heat conductance of the thermal insulation between the cap and the measurement subject.

Having described exemplary embodiments of the present invention it will be appreciated that variations on the described embodiments can be made. The present invention is not to be limited by the above description but rather by the appended claims.

1. A neurological imaging apparatus comprising:
a temperature-controlled chamber;
a plurality of sensing devices accommodated in the temperature-controlled chamber;
a head unit detachably mountable to the temperature-controlled chamber;
a plurality of pick-up devices for picking up respective measurement signals mounted in the head unit; and
an interface for communicating the measurement signals to the sensing devices.

2. A neurological imaging apparatus according to claim 1 wherein the sensing devices are superconducting devices, e.g. SQUIDS, HyQUIDs or Andreev interferometers.

3. A neurological imaging apparatus according to claim 1 wherein the pick-up devices are pick-up coils.

4. A neurological imaging apparatus according to claim 1 wherein the interface comprises a plurality of transmitter coils in the head unit and connected to respective ones of the sensing devices; and a plurality of receiver coils in the temperature-controlled chamber and coupled to the sensing devices; wherein each transmitter coil is configured to have a mutual inductance with a respective one of the receiver coils when the head unit is mounted on the temperature-controlled chamber.

5. A neurological imaging apparatus according to claim 4 wherein the pick-up coils, transmitter coils, receiver coils and interconnections between them are superconducting when the apparatus is operational.

6. A neurological imaging apparatus according to claim 1 further comprising a second head unit, the second head unit having a second plurality of pick-up devices, the second plurality of pick-up devices being differently configured from the plurality of pick-up devices.

7. A neurological imaging apparatus according to claim 6 wherein the pick-up devices are configured to confirm to a first body part of a measurement subject and the second pick-up devices are configured to conform to a second body part of a measurement subject, the second body part being different than the first body part.

8. A neurological imaging apparatus according to claim 7 wherein the first and second body parts are heads of different sizes.

9. (canceled)

10. A neurological imaging apparatus according to claim 6 wherein the plurality of pick-up devices are configured to conform to a body part when at a first orientation relative to the apparatus and the second plurality of pick-up devices are configured to conform to the body part when at a second orientation relative to the apparatus.

11. A neurological imaging apparatus comprising:
a temperature-controlled chamber;
a plurality of sensing devices accommodated in the temperature-controlled chamber; and
an interface for receiving a detachable head unit and communicating measurement signals therefrom to the sensing devices.

12. A head unit comprising a plurality of pick-up devices and configured for use with an apparatus according to claim 1.

13. A neurological imaging method using a neurological imaging apparatus having detachable head units, the method comprising:
selecting a head unit suitable for imaging a body part of a measurement subject;
mounting the head unit to the neurological imaging apparatus; and
imaging the body part of the measurement subject.

14. A neurological imaging method according to claim 13 wherein the sensing devices are superconducting devices, e.g. SQUIDS, HyQUIDs or Andreev interferometers.

15. A neurological imaging method according to claim 13 wherein the pick-up devices are pick-up coils.

16. A neurological imaging method according to claim 13, wherein the interface comprises a plurality of transmitter coils in the head unit and connected to respective ones of the sensing devices; and a plurality of receiver coils in the temperature-controlled chamber and coupled to the sensing devices; wherein each transmitter coil is configured to have a mutual inductance with a respective one of the receiver coils when the head unit is mounted on the temperature-controlled chamber.

17. A neurological imaging method according to claim 16 wherein the pick-up coils, transmitter coils, receiver coils and interconnections between them are superconducting when the method is operational.

18. A neurological imaging method according to claim 13 further comprising a second head unit, the second head unit having a second plurality of pick-up devices, the second plurality of pick-up devices being differently configured from the plurality of pick-up devices.

19. A neurological imaging method according to claim 18 wherein the pick-up devices are configured to confirm to a first body part of a measurement subject and the second pick-up devices are configured to conform to a second body part of a measurement subject, the second body part being different than the first body part.

20. A neurological imaging method according to claim 19 wherein the first and second body parts are heads of different sizes.

21. (canceled)

22. A neurological imaging method according to claim 18 wherein the plurality of pick-up devices are configured to conform to a body part when at a first orientation relative to the apparatus and the second plurality of pick-up devices are configured to conform to the body part when at a second orientation relative to the apparatus.

23. (canceled)
